(19) United States
(12) Patent Application Publication
(10) Pub. No.: US 2026/0000321 A1
(43) Pub. Date: Jan. 1, 2026

(54) DATA VISUALIZATION FOR CONTINUOUS GLUCOSE MONITORING

(71) Applicant: Allez Health Inc., Carlsbad, CA (US)

(72) Inventors: Hubert Hausegger, Encinitas, CA (US); Eli Reihman, San Diego, CA (US); Leif Bowman, San Diego, CA (US)

(73) Assignee: Allez Health Inc., Carlsbad, CA (US)

(21) Appl. No.: 19/251,451

(22) Filed: Jun. 26, 2025

Related U.S. Application Data
(60) Provisional application No. 63/666,410, filed on Jul. 1, 2024.

(51) Int. Cl. A61B 5/145; A61B 5/00

(52) U.S. Cl. CPC A61B 5/14532; A61B 5/7282; A61B 5/743; A61B 5/14865

(57) ABSTRACT

A method for continuous glucose monitoring includes a processor of a computing device operatively coupled to or integrated with a display receiving data. The data includes a plurality of glucose levels, and each glucose level of the plurality of glucose levels is associated with a corresponding time. The processor processes the data to generate a first graphical element indicative of a current glucose level, and one or more second graphical elements indicative of a rate and a direction of change of the glucose level. The processor via a graphical user interface displays the first graphical element on the display and the one or more second graphical elements on the display. The one or more second graphical elements are positioned relative to the first graphical element to visually indicate whether the glucose level is stable, increasing, or decreasing.

### RELATED APPLICATIONS

This application claims priority to U.S. Provisional Patent Application No. 63/666,410 filed on Jul. 1, 2024, which is hereby incorporated by reference in full.

### BACKGROUND

Medical patients often have diseases or conditions that require the measurement and reporting of biological conditions. For example, if a patient has diabetes, it is important that the patient have an accurate understanding of the level of glucose in their system. Traditionally, diabetes patients have monitored their glucose levels by sticking their finger with a small lance, allowing a drop of blood to form, and then dipping a test strip into the blood. The test strip is positioned in a handheld monitor that performs an analysis on the blood and visually reports the measured glucose level to the patient. Based upon this reported level, the patient makes important decisions on what food to consume, or how much insulin to inject. Diabetes is a devastating disease that if not properly controlled can lead to detrimental physiological conditions such as kidney failure, skin ulcers, bleeding in the eyes and eventually blindness, and pain and the eventual amputation of limbs.

Blood glucose levels are subject to rapid fluctuations caused by a variety of physiological, behavioral, and environmental factors, which can complicate accurate and timely glucose monitoring. Accordingly, a single glucose measurement provides only a snapshot of the instantaneous level in a patient's body. Such a single measurement provides little information about how the patient's use of glucose is changing over time, or how the patient reacts to specific dosages of insulin. Even a patient that is adhering to a strict schedule of strip testing will likely be making incorrect decisions as to diet, exercise, and insulin injection. This is exacerbated by a patient that is less consistent on their strip testing. To give the patient a more complete understanding of their diabetic condition and to get a better therapeutic result, some diabetic patients are now using continuous glucose monitoring.

Monitoring of glucose levels is critical for diabetes patients. Continuous glucose monitoring (CGM) sensors are a type of device in which glucose is measured from fluid sampled in an area just under the skin multiple times a day. CGM devices typically involve a small housing in which the electronics are located, and which is adhered to the patient's skin to be worn for a period of time. A small needle within the device delivers the subcutaneous sensor which is often electrochemical. Depending upon the patient's condition, continuous glucose monitoring may be performed at different intervals. For example, some continuous glucose monitors may be set to take multiple readings per minute, whereas in other cases the continuous glucose monitor can be set to take readings every hour or so. Electrochemical glucose sensors operate by using electrodes which typically detect an amperometric signal caused by oxidation of enzymes during conversion of glucose to gluconolactone. The amperometric signal can then be correlated to a glucose concentration.

A continuous glucose monitor has two main components. First, there is a housing for the electronics, processor, memory, wireless communication, and power. The housing is typically reusable over extended periods of time, such as months. This housing then connects or communicates to a disposable CGM sensor that is adhered to the patient's body, which typically uses an introducer needle to subcutaneously insert the sensor into the patient. CGM systems provide real-time glucose level data to patients with diabetes by measuring glucose levels in interstitial fluid. The sensor collects glucose data at regular intervals, and this data is then wirelessly transmitted to an external computing device, such as a smartphone or dedicated receiver, using various wireless communication protocols including BLUETOOTH®, near field communication (NFC), or other low-power wireless technologies.

The transmitted data is processed and displayed on the computing device via an application, providing users with immediate and continuous access to their glucose levels. This wireless data transmission allows for continuous monitoring without the need for manual readings, enhancing convenience and compliance. The ability to track glucose levels in real-time enables more precise management of diabetes, potentially reducing the risk of hyperglycemia and hypoglycemia. Moreover, the historical data collected can be used by healthcare providers to adjust treatment plans more effectively.

### SUMMARY

In aspects, a method for continuous glucose monitoring includes a processor of a computing device operatively coupled to or integrated with a display receiving data. The data includes a plurality of glucose levels, and each glucose level of the plurality of glucose levels is associated with a corresponding time. The processor processes the data to generate a first graphical element indicative of a current glucose level, and one or more second graphical elements indicative of a rate and a direction of change of the glucose level. The processor via a graphical user interface displays the first graphical element on the display and the one or more second graphical elements on the display. The one or more second graphical elements are positioned relative to the first graphical element to visually indicate whether the glucose level is stable, increasing, or decreasing.

In aspects, a system for continuous glucose monitoring includes a computing device operatively coupled to or integrated with a display, and having a processor and memory. The processor is configured to execute instructions to receive data comprising a plurality of glucose levels. Each glucose level of the plurality of glucose levels is associated with a corresponding time. The data is processed to generate a first graphical element indicative of a current glucose level, and one or more second graphical elements indicative of a rate and a direction of change of the glucose level. The first graphical element is displayed via a graphical user interface on the display. The one or more second graphical elements are displayed via the graphical user interface on the display. The one or more second graphical elements are positioned relative to the first graphical element to visually indicate whether the glucose level is stable, increasing, or decreasing.

### BRIEF DESCRIPTION OF THE DRAWINGS

FIG. 1A is a system for continuous glucose monitoring, in accordance with some aspects.

FIG. 1B is a front view of a display of a computing device with a graphical user interface for glucose monitoring, in accordance with some aspects.

FIGS. 2A-2C are front views of a display of a computing device with graphical user interfaces for glucose monitoring, in accordance with some aspects.

FIGS. 3A-3G are front views of displays of a computing device with graphical user interfaces for glucose monitoring, in accordance with some aspects.

FIGS. 4A-4C are front views of a display of a computing device with a graphical user interface for glucose monitoring, in accordance with some aspects.

FIG. 5 is a flowchart representing a method for data visualization for continuous glucose monitoring, in accordance with some aspects.

FIGS. 6A-6I are front views of images in a sequence for a display or portion thereof with a transitional graphical user interface for glucose monitoring, in accordance with some aspects.

FIGS. 7A-7D show front views of a display or portion thereof with graphical user interfaces for glucose monitoring, in accordance with some aspects.

FIGS. 8A-8F show front views of a display or portion thereof with graphical user interfaces for glucose monitoring, in accordance with some aspects.

FIGS. 9A-9C are front views of a display or portion thereof with graphical user interfaces for glucose monitoring, in accordance with some aspects.

### DETAILED DESCRIPTION

Tracking glucose levels with a Continuous Glucose Monitor (CGM) provides valuable data that can significantly enhance diabetes management. For example, by analyzing the CGM data, users can determine the percentage of time their glucose levels remain within the target range, known as Time in Range (TIR). This metric helps in understanding overall glucose control and identifying patterns in glucose fluctuations. Additionally, the data can be used to calculate Time Above Range (TAR), also referred to as high glucose level, and Time Below Range (TBR), also referred to as low glucose level, indicating periods of hyperglycemia and hypoglycemia, respectively. This information is necessary for making informed adjustments to medication, diet, and lifestyle to improve glycemic control.

In some aspects, a method for continuous glucose monitoring includes the use of a processor of a computing device that is operatively coupled to or integrated with a display. The processor receives data including a plurality of glucose levels, each of which is associated with a corresponding time. The processor processes the data to generate a first graphical element that represents the user's current glucose level, and one or more second graphical elements that visually communicate the rate and direction of change of the glucose level over time.

In some aspects, the processor displays these graphical elements on the display via a graphical user interface. The first graphical element may be rendered in a visually prominent form, such as a color-coded circle, to indicate whether the glucose level is within, above, or below a user-defined target range. The one or more second graphical elements are positioned relative to the first graphical element to visually indicate whether the glucose level is stable, increasing, or decreasing. For example, the second graphical elements may appear on each side of the first graphical element when the glucose level is stable, on one side (e.g., above or to the right) the first graphical element when the glucose level is increasing, or on an opposite side (e.g., below or to the left) of the first graphical element when the glucose level is decreasing.

In some aspects, the display of the second graphical elements also conveys the rate and severity of glucose change. This is achieved not only by the number and positioning of these elements but also through animation effects that simulate motion. The system calculates the first derivative of the glucose data to determine the rate of change, and the second derivative to determine the acceleration of that change. Acceleration refers to how quickly the rate itself is increasing or decreasing. For example, if the glucose level is rising and the rate of rise is increasing, this may be classified as very rapidly rising.

To visually communicate this information, the system may use animation properties such as pulsing speed, brightness, spacing, and simulated spring-like movement. These visual cues create a cascading effect, where one graphical element appears to push or pull the next, creating a ripple-like flow. This flow represents continuity and direction in the glucose trend and helps the user intuitively understand the urgency or severity of the trend. For example, a rapid acceleration in glucose levels may cause faster or more intense animation, drawing the user's attention to the need for immediate action. In contrast, minimal or slow animation may indicate a gradual trend that may not require immediate intervention but still warrants observation. When no animation is present, it may indicate that the glucose level is stable.

This system provides several advantages. It enables users to quickly and intuitively interpret their glucose data without the need to study numerical trends or raw charts. The use of color, shape, motion, and position simplifies complex data into an easily recognizable visual summary. This approach is particularly useful for small-screen devices such as smartwatches, where space is limited. Additionally, by showing not just the direction but also the acceleration of glucose trends, the system supports more informed and timely decision-making in glucose management. The visual effects are designed to enhance user awareness without overwhelming the interface, offering a balance of clarity and clinical insight.

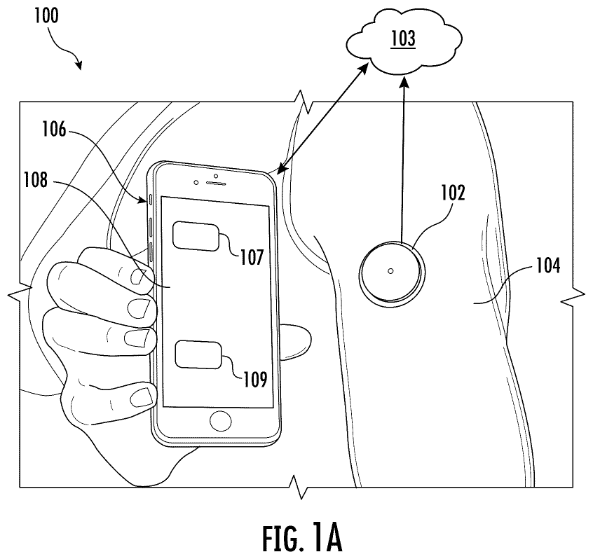

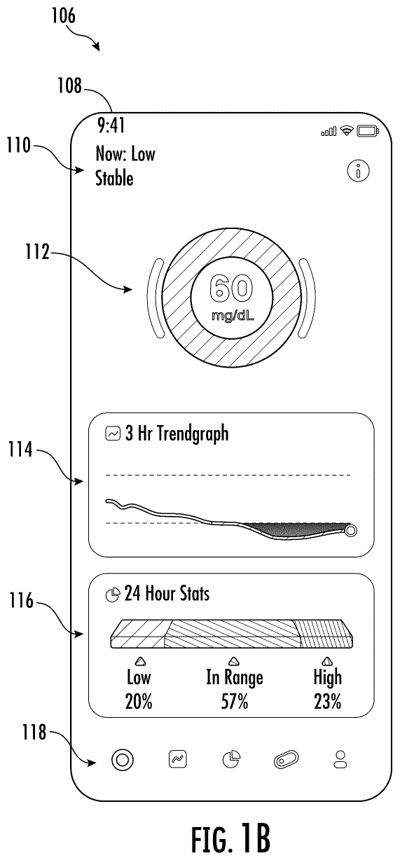

FIG. 1A is a system for continuous glucose monitoring, in accordance with some aspects. In some aspects, a system 100 includes a sensor 102, computing device 106, memory 107, display 108 and at least one processor 109. The sensor 102 is coupled to user 104 (e.g., patient). The sensor 102 may be a continuous glucose monitoring sensor, and may be applied subcutaneously, transdermally, transcutaneously, including implantable or other variations. The sensor 102 collects glucose data, such as glucose concentration levels (e.g., glucose levels) from the user's interstitial fluid, at either fixed or variable intervals. The data is wirelessly transmitted by way of a network 103 to the system 100. The network 103 generally represents any appropriate combination of the Internet, cell phone communication systems, broadband cellular networks, wide area networks (WANs), local area networks (LANs), wireless networks, networks based on the IEEE 802.11 family of standards (Wi-Fi networks), and other data communication networks.

The computing device 106 is configured to communicate using various wireless communication protocols, including BLUETOOTH®, near-field communications (NFC), or other wireless (e.g., low-power wireless) technologies. The computing device 106 may comprise a smartphone, smartwatch, tablet, dedicated receiver, or other suitable electronic device, and is operatively coupled to or integrated with the display 108 and the at least one processor 109 configured to receive and process data from the sensor 102. The computing device 106 may include the memory 107. In some aspects, the computing device 106 may operate an application configured to receive, process and display glucose-related data.

FIG. 1B is a front view of a display of a computing device with a graphical user interface for glucose monitoring, in accordance with some aspects. The display 108 of the computing device 106 presents a graphical user interface (GUI) for data visualization for glucose data from the continuous glucose monitoring via the sensor 102. The graphical user interface enables interaction between a user and the computing device 106 through visual indicators and graphical elements (such as icons, buttons, menus, windows, and other visual components) displayed on the display 108. The graphical user interface may support user input via touch, gesture, mouse, keyboard, or voice commands. In one example, the graphical user interface has a dashboard configuration illustrated on the display 108 showing a now (i.e., current) status 110, value trend indicator 112, trend graph 114, time in-range 116 and menu bar 118. The dashboard configuration presents the user 104 necessary data to quickly, easily and clearly visually understand their personal glucose data. In some cases, the user may select items from the menu bar 118 or directly from the display 108 to access additional displays (e.g., screens) that provide more detailed and/or targeted information.

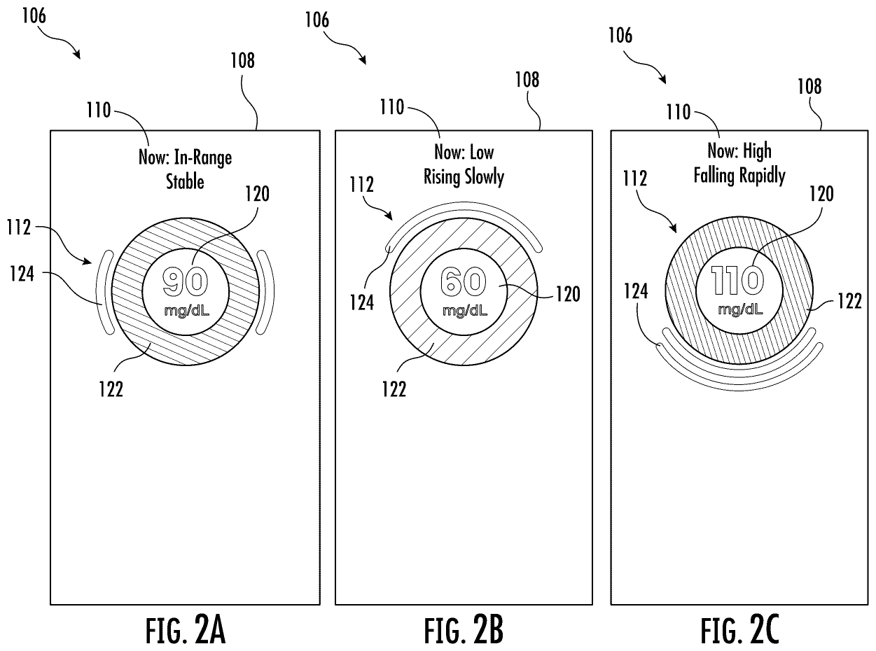

FIGS. 2A-2C are front views of a display of a computing device with graphical user interfaces for glucose monitoring, in accordance with some aspects. The now status 110 may be updated at predetermined intervals, such as every 10 seconds, 30 seconds, 60 seconds, three minutes, or similar durations. In some aspects, the user defines and sets a predefined target glucose range (e.g., in-range glucose level). This may be a range such as 90 to 130 mg/dL, 80 to 150 mg/dL or 70 to 160 mg/dL. The now status 110 includes two parts-a primary now status reflecting the actual value, and a secondary now status reflecting a rate of change. Both parts represent the current or now state of the user's glucose level from the connected sensor 102. For example, the primary now status of the now status 110 may be stable (TIR) as in FIG. 2A, low (TBR) as in FIG. 2B, or high (TAR) as in FIG. 2C, indicating the current glucose level of the user relative to the predefined target glucose range. The secondary now status of the now status 110 may be stable (FIG. 2A), falling (also referred to as decreasing), rising (or “rising slowly” as in FIG. 2B, also referred to as increasing), rapidly falling (FIG. 2C), rapidly rising, very rapidly falling, or very rapidly rising indicating the current trend of the glucose level.

The value trend indicator 112 is a visual representation of the now status 110. The value trend indicator 112 includes a glucose level 120, first graphical element 122 and second graphical element 124. The first graphical element 122 is an icon rendered in a color or pattern indicative of whether the glucose level is within the predefined target glucose range, greater than the predefined target glucose range, or less than the predefined target glucose range. In some examples, the icon may be represented by a circle, annular ring, oval, triangle, square, or any other shape suitable for conveying glucose status information. In this example, the first graphical element 122 is depicted as a circle, and the glucose level 120 is also displayed (90 mg/dL in FIG. 2A, 60 mg/dL in FIG. 2B, and 110 mg/dL in FIG. 2C in these examples). In some examples, the glucose level 120 is displayed within or adjacent to the first graphical element 122, such as positioned inside the circle, and is prominently presented to the user. The first graphical element 122 visually indicates the glucose level in relation to a predefined target glucose range.

The first graphical element 122 may be visually modified such as by shading, patterning, coloring, to indicate the current glucose level relative to the predefined target glucose range. In some examples, the first graphical element 122 may be shaded green to indicate that the current glucose level is within the predefined target glucose range (i.e., in-range). The first graphical element 122 may be shaded blue to indicate that the current glucose level is below the predefined target glucose range, or shaded red to indicate that the current glucose level is above the predefined target glucose range. The visual modification is an intuitive coding mechanism, providing a visual cue that enables users to quickly assess their glucose level/status at a glance. In some aspects, this information may be recorded over time to calculate metrics such as Time In-Range (TIR), Time Below Range (TBR), and Time Above Range (TAR), which may be generated for display over a selected duration.

The one or more second graphical elements 124 may comprise one or more elements that visually indicate the secondary now status of the now status 110. In some examples, the one or more second graphical elements are one or more arcs, bars, lines, or any other shape suitable for conveying glucose status information. The one or more second graphical elements 124 are displayed in relation to the first graphical element 122. A quantity of the one or more second graphical elements 124 corresponds to a calculated rate of change of the glucose level over time based on the collected data by the sensor 102. For instance, a greater quantity of second graphical elements indicates a faster rate of change. The position of the arcs 124 relative to the first graphical element 122 signifies the direction of glucose level change over time, providing users with a clear visual representation of both the speed and direction of their glucose levels over time or trends. In some examples, the one or more second graphical elements 124 are positioned on opposite sides of the first graphical element 122 to indicate that the glucose level is stable, positioned above the first graphical element 122 to indicate that the glucose level is increasing, or positioned below the first graphical element 122 to indicate that the glucose level is decreasing. In some examples, FIG. 2B has one arc for a slow rate of change (of the rising), and FIG. 2C has two arcs for a greater rate of change (of the falling).

In some aspects, a stable glucose level may be visually indicated by a geometric shape, such as a circle, rendered around the first graphical element 122. In some aspects, the first graphical element 122 may consist solely of the glucose reading 120 presented as text, without any surrounding shape. The text may be visually modified, such as through changes in color, shading, size, or brightness, to indicate the current glucose level relative to a predefined target glucose range. In other aspects, the first graphical element 122 may include descriptive words such as “HIGH,” “LOW,” or “IN RANGE” to convey the glucose level status. These textual indicators may also be visually modified in accordance with the methods described herein to enhance visibility and provide an intuitive visual cue to the user regarding their current glucose condition.

In other aspects, the one or more second graphical elements 124 may have multiple elements (e.g., arcs, lines, bars) which may be uniform in length, width, and appearance or may vary progressively. For example, each added element may be rendered slightly longer, thicker, or more prominent than the previous one. In other aspects, the one or more second graphical elements 124 may include trend arrows that pulsate or animate to visually indicate the presence and magnitude of glucose acceleration, as described herein. In yet other aspects, the second graphical elements 124 may be rendered as trend bars that dynamically fill upward or downward, or extend from left to right, to represent the direction and intensity of the glucose level change. These visual formats provide alternative representations to intuitively communicate glucose trends and rate-of-change information to the user.

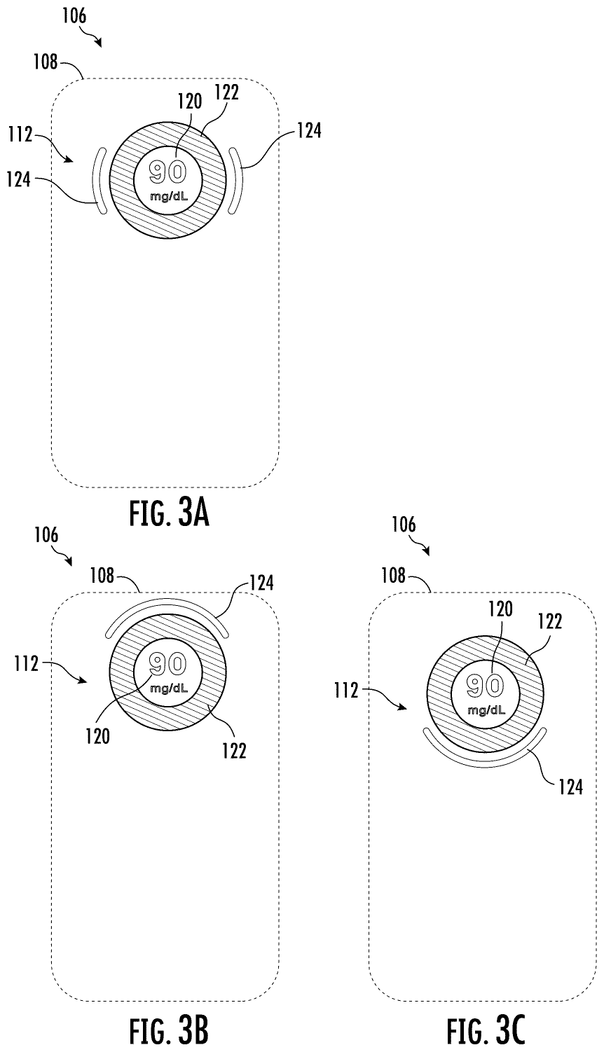

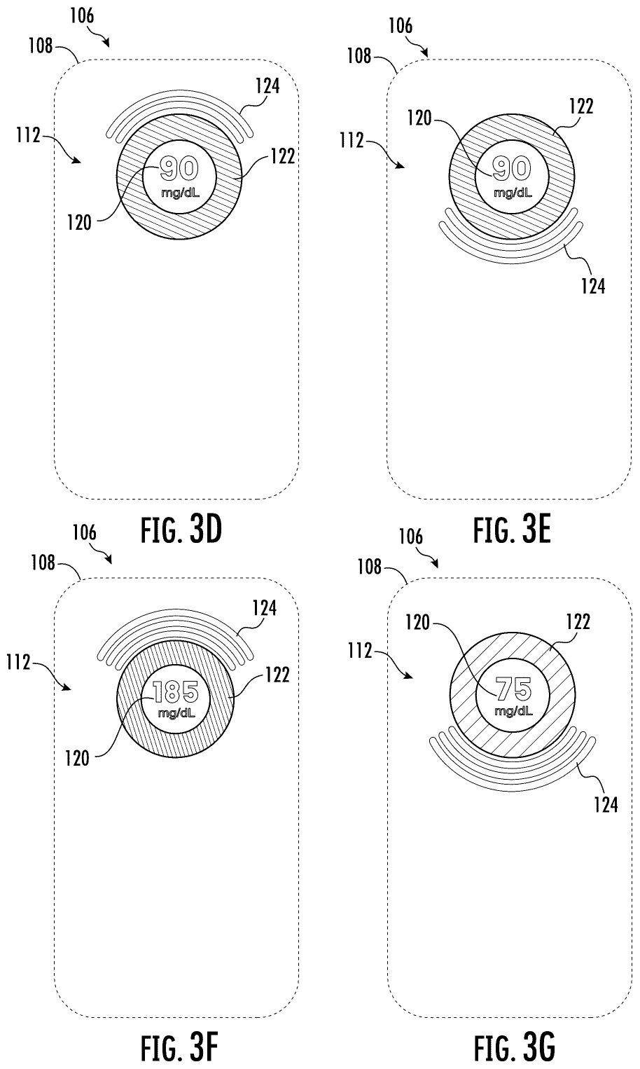

FIGS. 3A-3G are front views of displays of a computing device with graphical user interfaces for glucose monitoring, in accordance with some aspects. FIG. 3A illustrates the value trend indicator 112 with one second graphical element of the one or more second graphical elements 124 positioned on each side of the first graphical element 122, indicating that the glucose level is stable. FIG. 3B illustrates the value trend indicator 112 with one second graphical element of the one or more second graphical elements 124 above the first graphical element 122 indicating that the glucose level is rising, also referred to as increasing. FIG. 3C shows the value trend indicator 112 with the one second graphical element of the one or more second graphical elements 124 below the first graphical element 122 indicating that the glucose level is falling, also referred to as decreasing. FIG. 3D illustrates the value trend indicator 112 with two second graphical elements of the one or more second graphical elements 124 above the first graphical element 122 indicating that the glucose level is rapidly rising. FIG. 3E illustrates the value trend indicator 112 with two second graphical elements of the one or more second graphical elements 124 below the first graphical element 122 indicating that the glucose level is rapidly falling. In FIGS. 3A-3E, the first graphical element 122 may be visually modified with shading, patterns, color or another method, indicating that although the glucose levels may be changing, the glucose level is still in-range.

FIG. 3F illustrates the value trend indicator 112 with three second graphical elements of the one or more second graphical elements 124 above the first graphical element 122 indicating that glucose levels are very rapidly rising. As described, the first graphical element 122 may visually modified indicating that the glucose level is above range. In some cases, the color red may be used. In other examples, the glucose level may be in-range (e.g., the first graphical element 122 colored green) or below range (e.g., first graphical element 122 colored blue) but with a rate of change that is rising (e.g., one or more second graphical elements above the first graphical element 122).

FIG. 3G illustrates the value trend indicator 112 with three second graphical elements of the one or more second graphical elements 124 below the first graphical element 122 indicating that glucose levels are very rapidly falling. The first graphical element 122 in FIG. 3G may be colored blue, indicating that the glucose level is below range. In other examples, the glucose level may be in-range (e.g., first graphical element 122 colored green) or above range (e.g., first graphical element 122 colored red) but with a rate of change that is falling (e.g., one second graphical elements below the first graphical element 122), rapidly falling (e.g., two second graphical elements below the first graphical element 122) or very rapidly falling (e.g., three second graphical elements below the first graphical element 122).

This visualization of the value trend indicator 112 prioritizes spatial efficiency, ensuring that the form and shape make optimal use of the display 108. The compact and design of the one or more second graphical elements 124 allows for clear and immediate data representation, making it particularly effective for use on small screens, such as smartwatches. The configuration enhances the display of information within limited spaces while maintaining user accessibility and readability, making it useful for applications where screen size is a constraint.

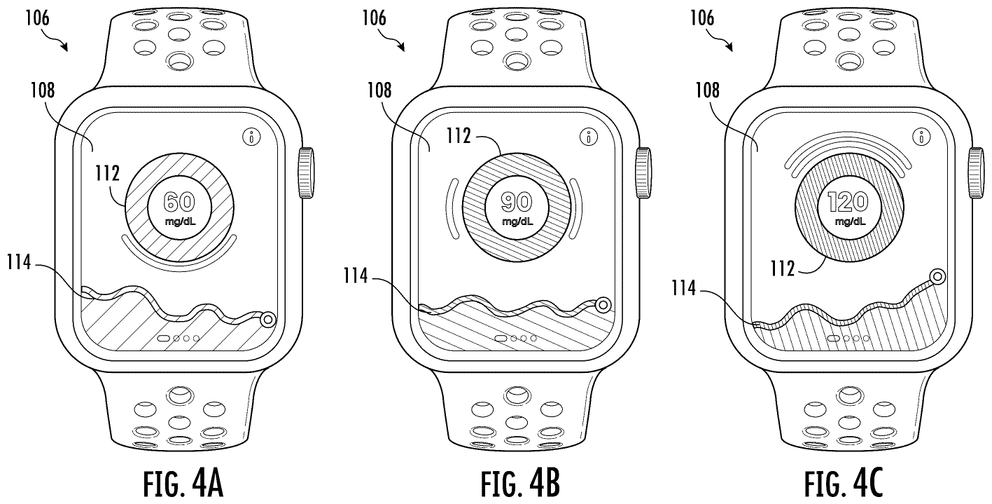

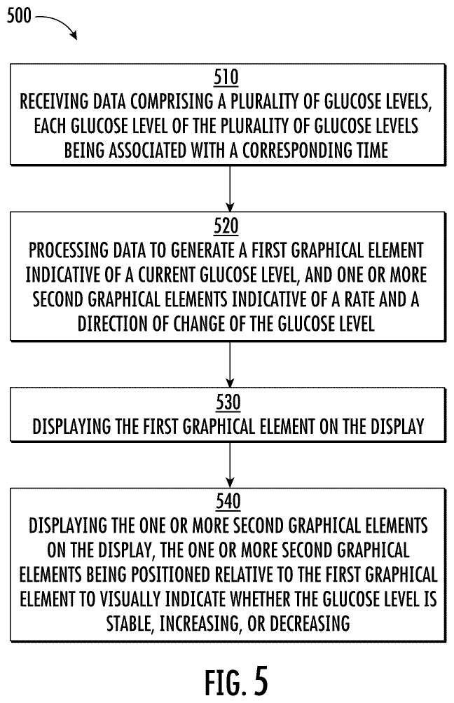

FIGS. 4A-4C are front views of a display of a computing device with graphical user interface for glucose monitoring, in accordance with some aspects. FIGS. 4A-4C show various configurations of the value trend indicator 112 on a computing device 106 with the display 108 such as a smartwatch. In some aspects, the trend graph 114 (described in later sections) is also displayed, further maximizing the limited available space of the display 108 of the smartwatch.

FIG. 5 is a flowchart representing a method for data visualization for continuous glucose monitoring, in accordance with some aspects. The particular steps, order of steps, and combination of steps are shown for illustrative and explanatory purposes only. Other embodiments can implement different particular steps, orders of steps, and combinations of steps to achieve similar functions or results. The method 500 for data visualization for continuous glucose monitoring starts at block 510. A computing device 106, such as a smartphone, smartwatch, tablet, dedicated receiver, or similar device is operatively coupled to or integrated with a display 108. A processor 109 of the computing device 106 receives data including a plurality of glucose levels. Each glucose level of the plurality of glucose levels is associated with a corresponding time. The data may be collected and transmitted from the sensor 102 continuously monitoring a glucose level of in-vivo glucose in body fluid of a patient or user 104.

At block 520, the processor 109 processes the data to generate a first graphical element 122 indicative of a current glucose level, and one or more second graphical elements 124 indicative of a rate and a direction of change of the glucose level. The processing further includes comparing the current glucose level to a predefined target glucose range. When the glucose level is within the predefined target glucose range, the glucose level is determined to be in-range of the predefined target glucose range. When the glucose level is less than the predefined target glucose range, the glucose level is determined to be below the predefined target glucose range, or low. When the glucose level is greater than the predefined target glucose range, the glucose level is determined to be above the predefined target glucose range, or high.

At block 530, the processor 109 via a graphical user interface displays the first graphical element 122 on the display 108. For instance, the first graphical element 122 may be shaded, patterned, colored or otherwise visually modified to indicate whether the glucose level is within the predefined target glucose range (e.g., in-range), greater than the predefined target glucose range (e.g., high), or less than the predefined target glucose range (e.g., low). In some examples, the processor 109 via a graphical user interface may display the glucose reading 120. This may be positioned within or adjacent to the first graphical element 122.

At block 540, the processor 109 of the computing device 106 via the graphical user interface, displays the one or more second graphical elements 124 on the display 108. The one or more second graphical elements 124 are positioned relative to the first graphical element 122 to visually indicate whether the glucose level is stable, increasing (or rising), or decreasing (or falling).

In some aspects, the processing further includes calculating a rate of change of the glucose level over a predefined time interval from the data. This calculated rate of change determines the positioning of the one or more second graphical elements 124. When the calculated rate of change is within a defined stability threshold range, the glucose level is determined to be stable, and the second graphical elements 124 are positioned on opposite sides of the first graphical element 122. When the calculated rate of change is greater than an upper threshold, the glucose level is determined to be increasing, and the second graphical elements 124 are positioned above the first graphical element 122. When the calculated rate of change is less than a lower threshold, the glucose level is determined to be decreasing, and the second graphical elements 124 are positioned below the first graphical element 122.

The processing further comprises generating a first derivative and a second derivative of the glucose level with respect to time. The first derivative represents the rate of change of glucose level over time (i.e., the speed at which the glucose level is increasing or decreasing or the speed at which the glucose level is rising or falling). The second derivative represents the acceleration of the glucose level, indicating how rapidly the rate of change itself is varying. For example, the second derivative may reflect whether the rate of increase in the glucose level is accelerating, whether the rate of decrease in the glucose level is decelerating, or whether other fluctuations in the rate of change are occurring. A magnitude of the glucose acceleration corresponds to an absolute value of this second derivative, independent of whether the glucose trend is upward or downward. The processor 109 may modify the quantity of the one or more second graphical elements 124 and animation characteristics based on the computed derivatives.

The one or more second graphical elements 124 may be animated using animation characteristics such as intensity, pulsing frequency, or motion speed. These animation characteristics may be adjusted by the processor based on the magnitude of the glucose acceleration to visually convey the urgency of a detected glucose level trend. In some aspects, a high magnitude of glucose acceleration results in a faster or more pronounced animation to indicate that rapid action by the user may be required. Conversely, a low magnitude of glucose acceleration may result in slower or minimal animation changes, visually indicating that the glucose level trend is changing gradually and may not require immediate user action. In some aspects, the absence of animation may indicate that the glucose level is stable or within the predefined target glucose range and no action is required.

The visual representation of glucose data utilizes the one or more second graphical elements 124 around a control element such as the first graphical element 122 to indicate real-time changes in glucose levels. In one example, as shown in FIG. 3D, two second graphical elements 124 are positioned above the first graphical element 122 to indicate that the glucose level is increasing. The quantity of the second graphical elements 124 corresponds to the calculated rate of change of the glucose level. In this example, the position above the first graphical element 122 visually indicates an upward trend, while the presence of two second graphical elements 124 signifies that the glucose level is rapidly increasing, as chosen from stable, increasing, rapidly increasing or very rapidly increasing. In some aspects, these classifications may be predefined by the manufacturer. In other cases, they may be configurable by a clinician, caregiver, or the user, allowing the system to be tailored to individual needs or clinical protocols. In another example, as shown in FIG. 3G, three second graphical elements 124 are positioned below the first graphical element 122 and indicate that the glucose level is very rapidly decreasing. The downward position relative to the first graphical element 122 visually communicates a decreasing glucose trend, while the quantity of second graphical elements 124 corresponds to the calculated rate of decrease, with a greater number indicating a faster rate of change. In this example, very rapidly decreasing is chosen from stable, decreasing, rapidly decreasing or very rapidly decreasing.

The method 500 may further include animating the one or more second graphical elements 124 in a sequential manner to visually represent glucose acceleration. The rate of the animation may indicate the rate at which glucose levels are changing, whether they are stable, increasing, decreasing, rapidly increasing, rapidly decreasing, very rapidly increasing, or very rapidly decreasing. The one or more second graphical elements 124 may be animated so that each second graphical element 124 moves in a sequential manner indicative of an acceleration in increasing or decreasing rate of change of glucose levels over time based on the data from the sensor 102. In other words, the one or more second graphical elements 124 may be animated by sequentially adjusting a position of each second graphical element 124 of the one or more second graphical elements 124 to vary spacing between adjacent second graphical elements. In some aspects, the one or more second graphical elements 124 are equally spaced from one another and from the first graphical element 122 when the glucose level is stable. The one or more second graphical elements 124 are repositioned and animated by the processor 109 in response to the calculated rate of change and acceleration of the glucose level.

The animation of the one or more second graphical elements 124 is indicative of a direction and a magnitude of a change in glucose levels over time. In some examples, the animation of the one or more second graphical elements 124 is a pulsing motion that moves each second graphical element of the one or more second graphical elements 124 in a sequential manner to indicate acceleration in rising or falling glucose levels. The pulsing rate of each second graphical element of the one or more second graphical elements 124 increases with an increasing magnitude of glucose acceleration. The faster the pulsing of the one or more second graphical elements 124, the faster the acceleration of the change in glucose levels. The one or more second graphical elements 124 are animated as sequentially shifting elements, each transitioning forward in time to represent changes in glucose level between successive data points.

In some examples, the animation of the one or more second graphical elements 124 can be described as a cascading sequence, in which the movement of one element initiates the subsequent movement of another, visually indicating a directional trend in glucose acceleration. The animation forms a dynamic flow among the one or more second graphical elements 124, with the movement resembling a spring-like or ripple effect. Each second graphical element 124 is animated to simulate spring-like behavior, such that a first shifting element appears to push or pull an adjacent shifting element, visually conveying continuity in the direction of movement associated with the glucose trend. The second graphical elements 124 are virtually connected, with each element influencing the next in a sequential manner to produce the cascading effect. In some examples, the animation effect of the one or more second graphical elements 124 may involve vertical movement, such as an up-and-down oscillation relative to the first graphical element 122. In other examples, the animation may involve horizontal movement, where the arcs shift side to side to create a wave-like or swaying effect. These directional animations may be used independently or in combination.

In other examples, the one or more second graphical elements 124 the thickness of each graphical element may increase or decrease dynamically to reflect the severity or urgency of the trend. For example, a thicker arc may indicate a higher rate of change or greater acceleration in the glucose level. In other variations, the one or more second graphical elements 124 may simultaneously flash on and off at a defined pulsing rate, rather than animating sequentially. These variations may be used individually or in combination with other animation behaviors such as color changes, brightness modulation, or directional positioning.

In some examples, the animation continues based on the previously classified glucose trend, such as a determination that the glucose level is very rapidly rising, and persists until a new trend classification is determined. For example, if the glucose level is classified as very rapidly rising, the corresponding spring-like animation remains active to visually reinforce that trend classification. The animation is updated only after a new trend classification is confirmed, such as determining that the glucose level is now decreasing. This approach ensures visual continuity and reinforces the urgency of the identified trend without introducing abrupt or conflicting animation changes in response to minor or temporary fluctuations in the glucose data.

The method 500 improves the user's understanding of data trends through a noncomplicated and effective animated visualization, optionally enhanced by pulsing animations to emphasize the direction and magnitude of glucose level changes. The pulsing or animation of the one or more second graphical elements 124 demonstrates the acceleration of glucose levels. The present animated visualizations indicate in more detail, compared to conventional glucose monitoring systems, whether the user's blood sugar levels are rising or falling steadily, rapidly, or very rapidly. Knowing whether glucose levels are changing at a steady or rapid rate is critical additional data for effective diabetes management. This information can help patients and healthcare providers better predict and prevent dangerous swings in blood sugar levels. For instance, a rapid rise in glucose might necessitate an immediate adjustment in insulin dosage or dietary intake to avoid hyperglycemia, whereas a steady rise might require a more moderate response. Similarly, understanding a rapid fall in glucose can prompt quicker actions to prevent hypoglycemia. By distinguishing between steady and rapid changes, known as acceleration, users can make more precise and timely decisions about their treatment, leading to more stable blood sugar levels and reducing the risk of complications. This additional layer of data contributes to more personalized and effective diabetes management strategies.

The data visualization for continuous glucose monitoring shows speed and acceleration. Speed is indicated by the quantity of the one or more second graphical elements 124 displayed on the value trend indicator 112. Acceleration is shown by the animation of the one or more second graphical elements 124 such as intensity of pulsing of the one or more second graphical elements 124 (e.g., slow and fast). In some aspects, an intensity or a speed of the animating corresponds to a magnitude of the glucose acceleration. The processor 109 process data inputs including time, glucose levels, among others, to extract useful information, make decisions, and perform calculations for the data visualization of speed and acceleration of glucose levels. In some aspects, the animation is updated at fixed time intervals that correspond to receiving new glucose measurements from sensor 102, such as at intervals of approximately one minute, two minutes, five minutes, or any interval within a range between one and five minutes. The user may define a predefined target glucose range (also referred to as a desired in-range threshold) for the glucose level. From this, data such as glucose levels that are in-range and out of range (e.g., low or high) are recorded and stored. The data can be processed over a specific duration to determine the rate at which the glucose levels are changing over time. By analyzing the slope of a time versus glucose level curve, the rate of change can be used to adjust the speed of the animation accordingly. The rate of change may be measured in mg/dL/minute. In some aspects, one second graphical element of the one or more second graphical elements 124 of the value trend indicator 112 represents one mg/dL/minute per minute, two second graphical elements 124 represents two mg/dL/minute, and three second graphical elements 124 represents three mg/dL/minute (or more).

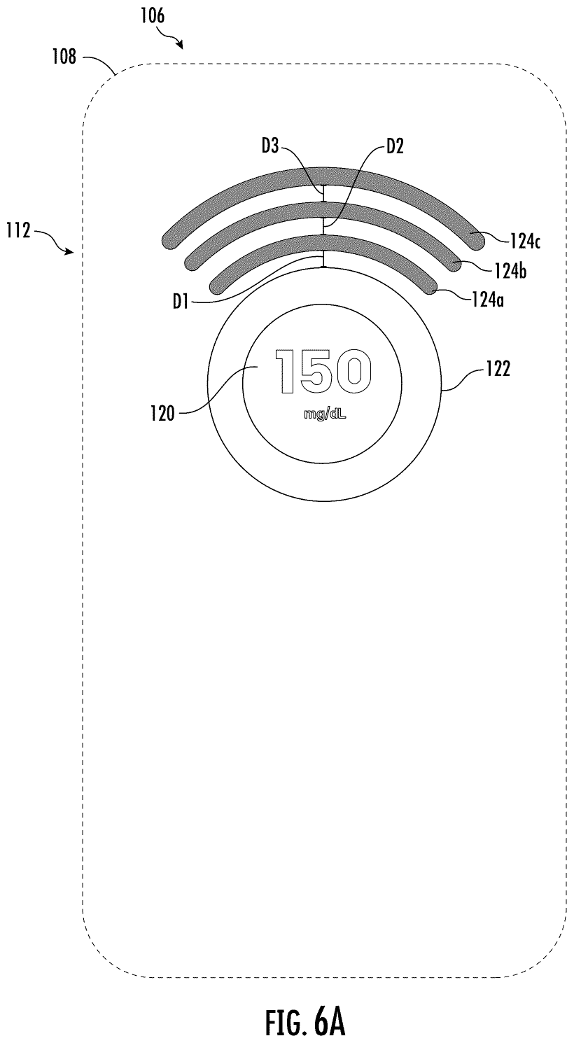

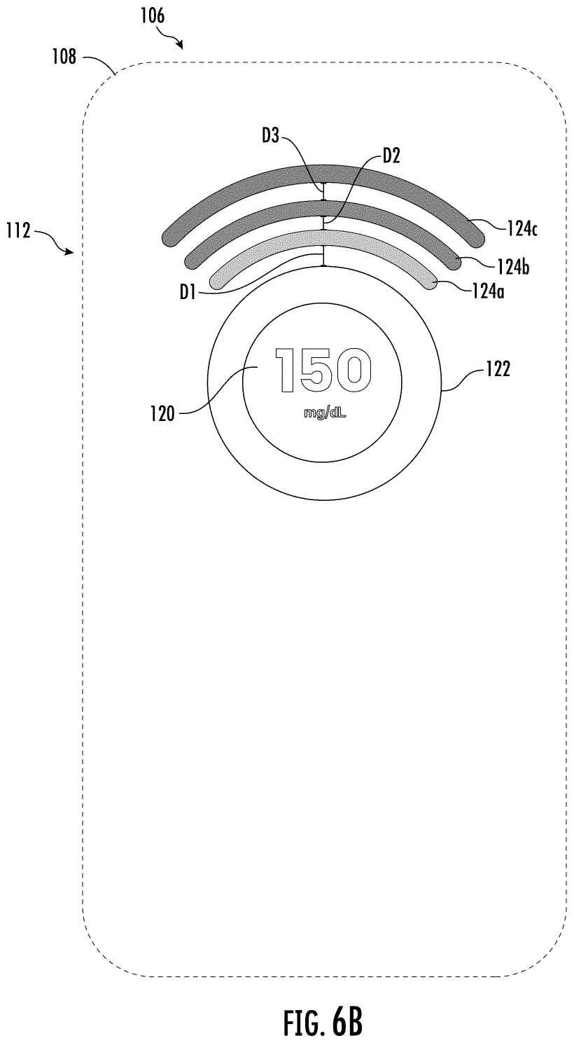

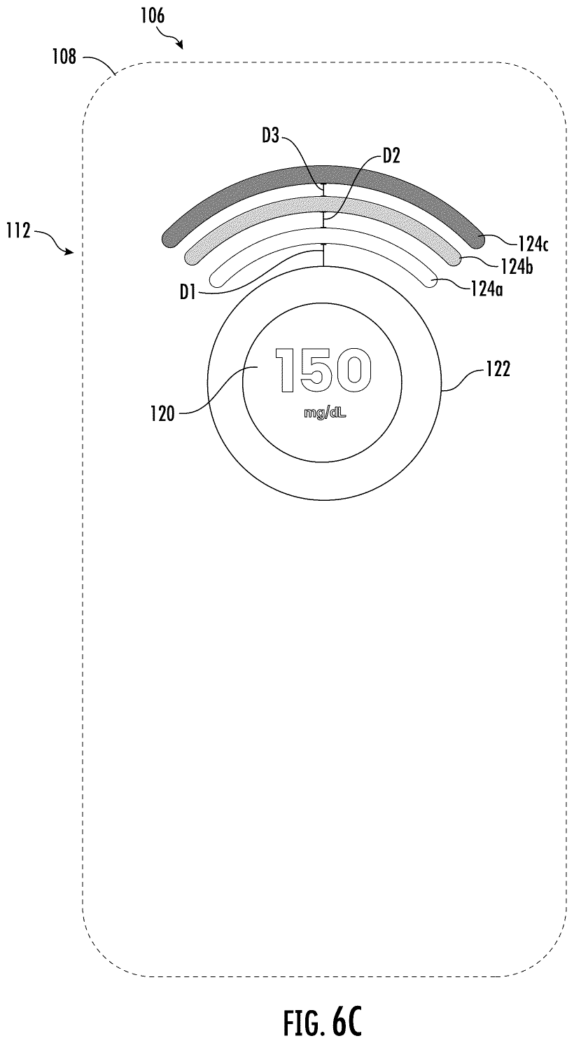

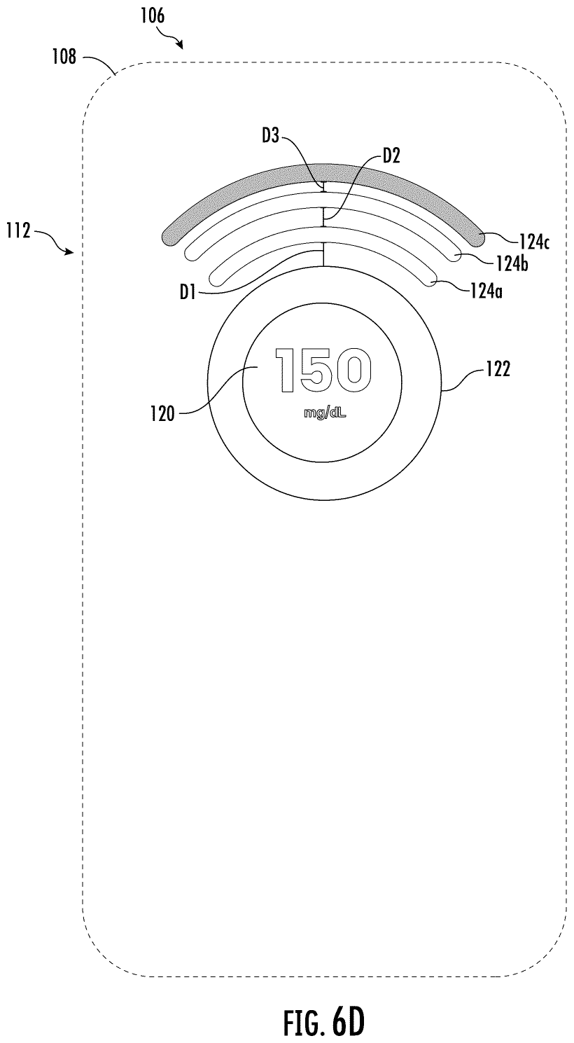

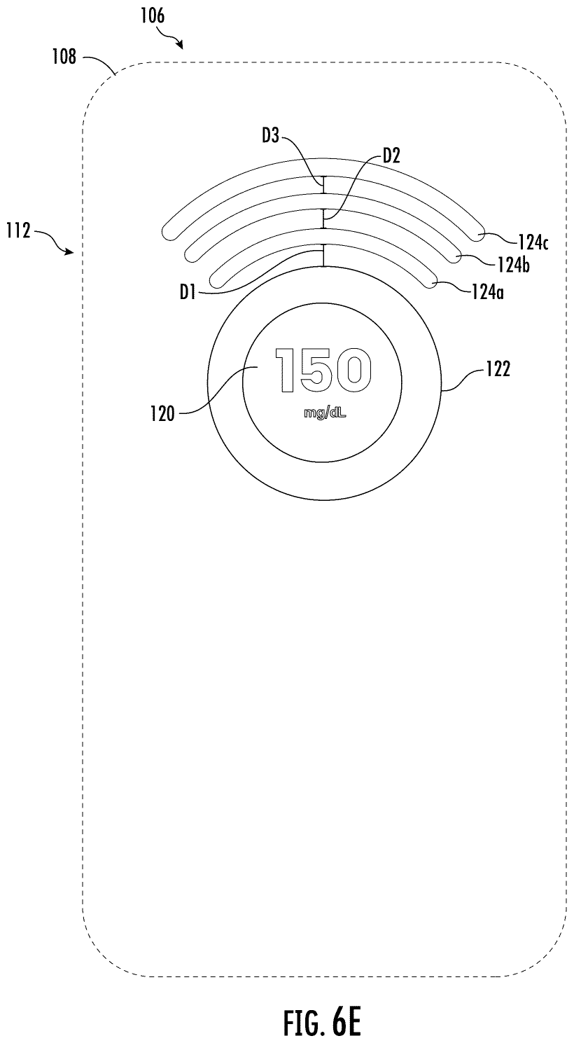

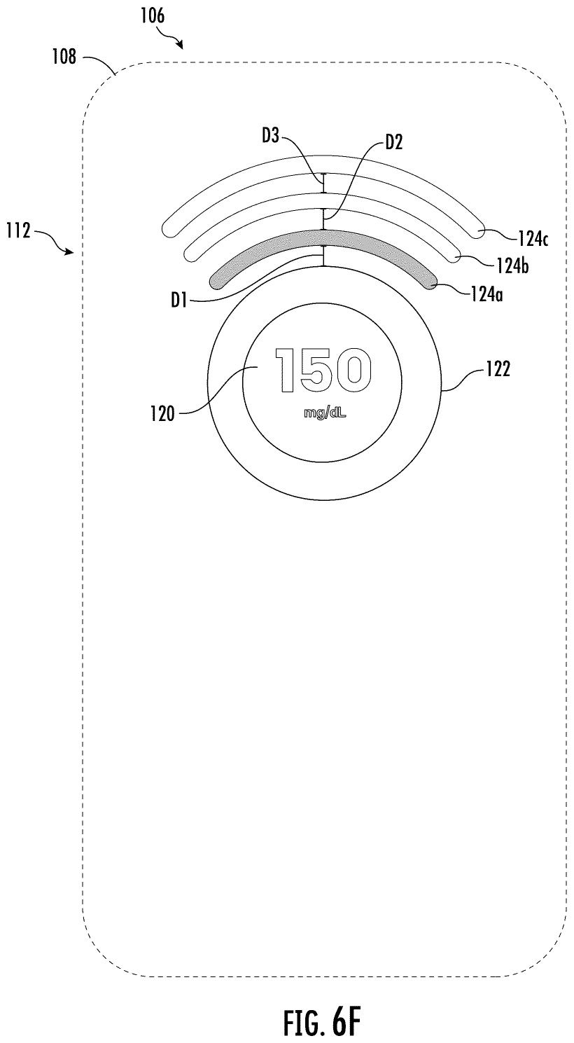

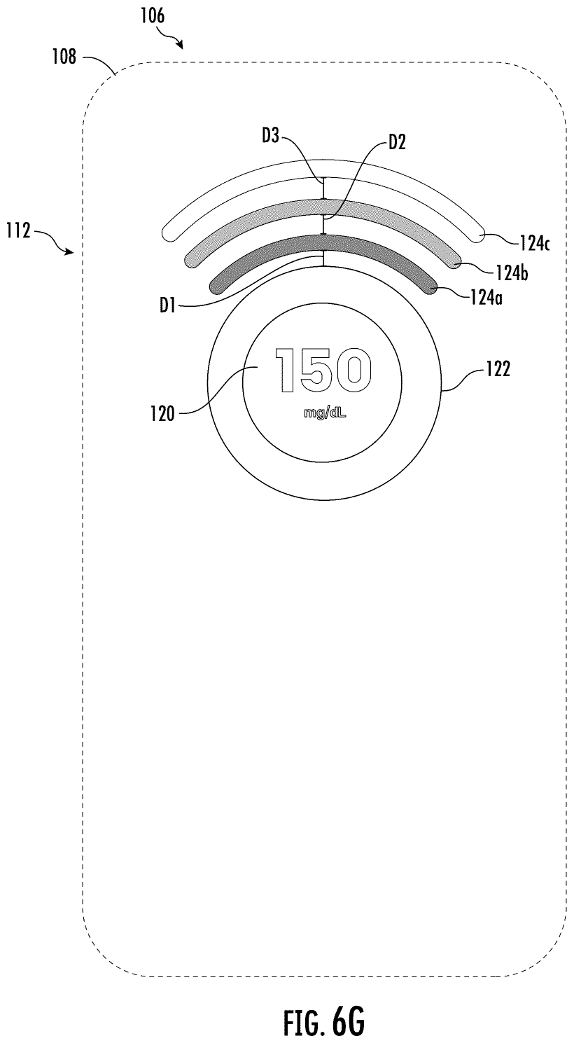

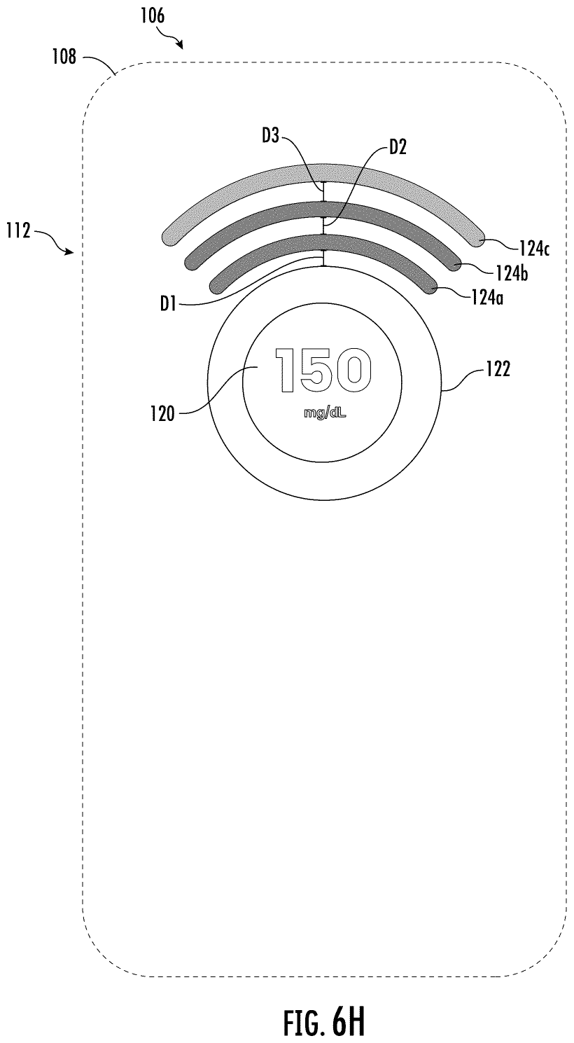

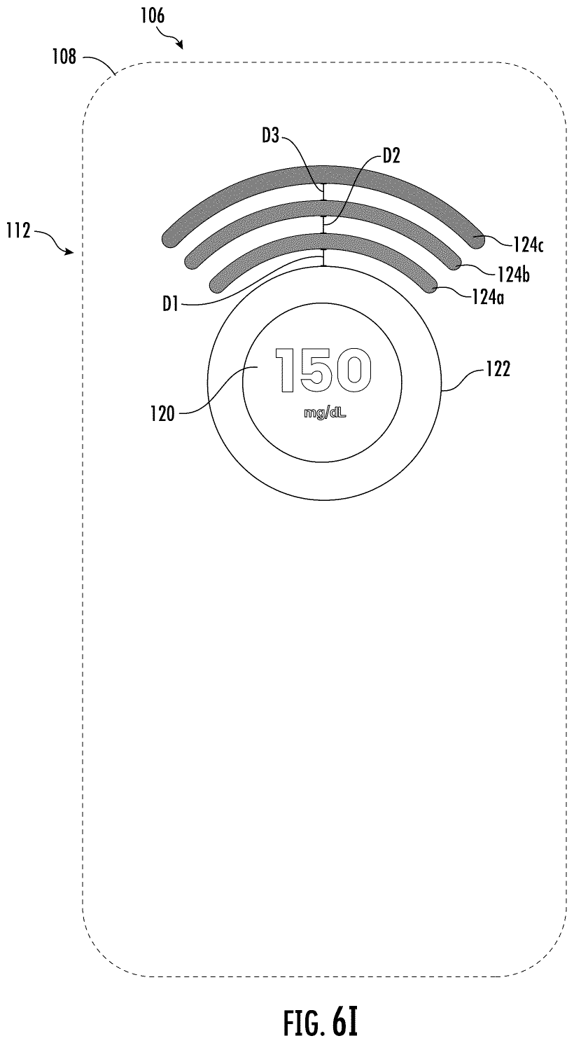

Referring to FIG. 3A, in some aspects, in a stable configuration, the one second graphical element of the one or more second graphical elements 124 is not animated. This is a visual cue to the user that no adjustments to the user's blood sugar levels are needed at the current time. Referring to FIGS. 3B-3F, the one or more second graphical elements 124 may be animated. FIGS. 6A-6I are front views of images in a sequence for a display 108 or portion thereof with a transitional graphical user interface for glucose monitoring, in accordance with some aspects. FIG. 6A shows the value trend indicator 112. The glucose reading 120 is shown as 150 mg/dL, and the first graphical element 122 may be shaded a color such as green indicating the glucose level is in-range for the user 104. The one or more second graphical elements 124 are positioned above the first graphical element 122 indicating the glucose level is rising and because the quantity of the one or more second graphical elements 124 is three, the glucose level is very rapidly rising.

In this example, the one or more second graphical elements 124 are illustrated as three arcs, 124a, 124b and 124c respectively, and are equally spaced apart from one another so that the distance between each arc is the same. That distance is the same distance between arc 124a and first graphical element 122. In one example, there may be three second graphical elements 124 positioned below the first graphical element 122 indicating the glucose level is falling. Because the quantity of second graphical elements 124 is three, the glucose level may be dropping by more than 3 mg/dL/minute, but the pulsing of the one or more second graphical elements 124 may be slower to communicate to user that the speed of change of the glucose level is slowing moving toward 3 mg/dL/minute. The slower pulsing rate of the one or more second graphical elements 124 is a visual cue to the user whether a change in state (e.g., consuming a particular food, exercise, etc.) mitigates the glucose level. The user can determine if the change in state is effective or not effective based on the pulse rate.

The one or more second graphical elements 124 may be animated by modifying a visual property of each second graphical element of the one or more second graphical elements 124 in a time-based sequence that simulates a spring-like or ripple motion away from or toward the first graphical element 122. The visual property may be chosen from color, spacing, pulsing rate, and brightness, or a combination thereof. In some aspects, the visual property of each second graphical element of the one or more second graphical elements 124 is transitioned or changed in a sequence to represent a trend in glucose level over time. In some examples, the transitioning or changing of the one or more second graphical elements 124 may be gradual or abrupt. The animating is controlled by the processor 109 based on one or more calculated metrics derived from the glucose data, including a rate of change in the glucose level and an acceleration of the glucose level.

In some examples, the transitioning or modification of the one or more second graphical elements 124 may occur in either a gradual or an abrupt manner. A gradual transition may involve smooth and continuous changes in visual properties such as color, brightness, spacing, position, or pulsing frequency over a defined time interval. This type of transition may visually communicate a slow or steady change in glucose trends. An abrupt transition may involve immediate or discrete changes in one or more visual properties, such as a sudden shift in position, a rapid change in color intensity, or an immediate onset of pulsing animation. This may be used to signal a significant or urgent change in the glucose level or its acceleration, helping the user quickly recognize the need for action. In some aspects, a gradual or abrupt transition may be based on the magnitude of the calculated rate of change or acceleration of the glucose level. For example, a minor increase in glucose level may result in a slow expansion and soft brightening of an arc, while a sharp spike may trigger a rapid pulsing and repositioning of multiple second graphical elements.

A grayscale shading and/or visual modification such as color of the one or more second graphical elements 124 represents a contrast in appearance. The appearance of the animated graphical user interface transitions sequentially between the images shown in FIGS. 6A-6I. Distances are labeled on FIGS. 6A-6I such as D1 is the distance between the first graphical element 122 and arc 124a, D2 is the distance between arc 124a and arc 124b, and D3 is the distance between arc 124b and arc 124c. FIG. 6A illustrates that distances D1, D2, and D3 are equal, indicating uniform spacing between the first graphical element 122 and each of the adjacent arcs. During these transitions, FIG. 6B shows arc 124a beginning to transition to a lighter shade (e.g., white) and move away from the first graphical element 122 approaching arc 124b. As a result, distance D1 increases and becomes greater than distances D2 and D3.

In FIG. 6C, arc 124a moves further away from first graphical element 122, becoming fully white, while arc 124b begins to transition to a lighter shade and move closer to arc 124c. At this stage, distance D1 continues to increase, while distance D2 decreases and distance D3 remains relatively unchanged. FIG. 6D depicts arc 124a at its maximum distance from the first graphical element 122, with D1 at its largest value. Arc 124a is fully white, and arc 124b is now also fully white and positioned closer to arc 124c than in FIG. 6C. At this point, D2, the distance between arc 124a and arc 124b, is smaller than D1, and D3, the distance between arc 124b and arc 124c, is the smallest of the three. In FIG. 6E, arc 124c becomes fully white and moves away from arc 124b. At this stage, arcs 124a, 124b, and 124c are all fully white and are approximately equally spaced, with distances D1, D2, and D3 being roughly equal. Arc 124a remains positioned at its furthest distance from the first graphical element 122, corresponding to the maximum value of D1.

The transitional sequence shown in FIGS. 6A-6E creates a ripple effect. The one or more second graphical elements 124 are animated in a ripple pattern consistent with a trend in glucose levels based on calculated acceleration. The animating is configured to move each second graphical element of the one or more second graphical elements 124 in a time-ordered ripple sequence that visually conveys a trend in acceleration of the glucose level, such as stable, increasing, decreasing, rapidly increasing, rapidly decreasing, very rapidly increasing or very rapidly decreasing. In some aspects, the one or more second graphical elements 124 transition sequentially to fade-to-bright and fade-to-dark across the one or more second graphical elements 124 during outward and return movements, respectively. Arc 124a fades to white and moves away from first graphical element 122, pushing or bumping arc 124b. In this context, “fades” means changing from a dark color to a light color such as gray to white. In other words, the arc is a dark color then brightens in color to white as a visual cue catching the user's glance.

As arc 124a fades and moves, arc 124b also begins to fade to white while moving away from arc 124a, subsequently pushing or bumping arc 124c. Finally, arc 124c fades to white and moves away from arc 124b until it reaches its maximum distance. Put another way, initially, arc 124a fades to white and moves away from first graphical element 122, initiating a chain reaction. This motion causes arc 124b to fade to white and shift away from arc 124a, which in turn influences arc 124c to undergo the same transformation. Each arc sequentially fades to white and moves outward, maintaining this pattern until arc 124c reaches its maximum distance from arc 124b.

The transitional sequence continues in FIGS. 6F through 6I, where the arcs move toward the first graphical element 122 and transition from white to dark. In FIG. 6F, arc 124a begins to transition to a darker shade, such as gray, and moves toward the first graphical element 122 and away from arc 124b. At this stage, D1 decreases, while D2 increases, and D3 remains relatively unchanged. In FIG. 6G, arc 124a moves closer to the first graphical element 122 than in FIG. 6F and is now fully gray. Arc 124b begins to transition to a darker shade and moves closer to arc 124a while moving away from arc 124c. At this point, D1 continues to decrease, D2 becomes smaller, and D3 increases as arc 124b moves away from arc 124c. FIG. 6H illustrates arc 124a having returned to its original position near the first graphical element 122, as shown in FIG. 6A, and is fully gray. Arc 124b is also fully gray and is positioned closer to arc 124a than in FIG. 6G. At this stage, D1 returns to its original value, D2 is shorter than D3, and D3 remains extended as arc 124c continues its previous position. Finally, in FIG. 6I, arc 124c becomes fully gray and moves closer to arc 124b. At this point, all three arcs 124a, 124b, and 124c are fully gray and equally spaced, with D1, D2, and D3 being approximately equal. This configuration corresponds to the original layout shown in FIG. 6A, completing the full visual cycle.

The animation creates a directional effect, making it appear as if the one or more second graphical elements 124 (e.g., arcs) are always moving in a single direction, such as upward when positioned above first graphical element 122 or downward when positioned below first graphical element 122. Additionally, the speed at which the one or more second graphical elements 124 move during the animation is correlated with the rate of change in glucose levels, reflecting rapid changes with faster movements. For example, an animation speed of the one or more second graphical elements 124 may be proportional to the glucose acceleration. In some aspects, one arc may increase in size to appear larger than other arcs at that moment, creating a visual effect that draws the user's attention. This perceived difference in size or movement is a visual illusion intended to enhance the user's perception of trend urgency, rather than representing a physical change in the underlying data.

Referring to FIGS. 1A and 1B, in some aspects, a system 100 for continuous glucose monitoring includes a computing device 106 operatively coupled to or integrated with a display 108, and having a processor 109 and memory 107. The memory 107 may store instructions and other data which are machine-readable medium. Machine-readable medium refers to any non-transitory computer program product, apparatus and/or device (e.g., magnetic discs, optical disks, memory, Programmable Logic Devices (PLDs)) used to provide machine instructions and/or data to the programmable processor 109, including a machine-readable medium that receives machine instructions.

The processor 109 is configured to execute instructions to receive data comprising a plurality of glucose levels. Each glucose level of the plurality of glucose levels is associated with a corresponding time. The data is processed to generate a first graphical element 122 indicative of a current glucose level, and one or more second graphical elements 124 indicative of a rate and a direction of change of the glucose level. The first graphical element 122 is displayed via a graphical user interface on the display 108. The one or more second graphical elements 124 are displayed via the graphical user interface on the display 108. The one or more second graphical elements 124 are positioned relative to the first graphical element to visually indicate whether the glucose level is stable, increasing, or decreasing.

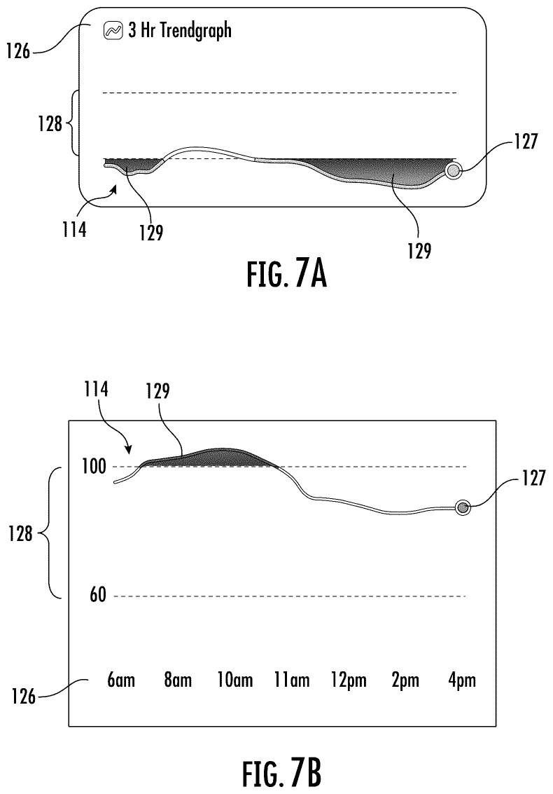

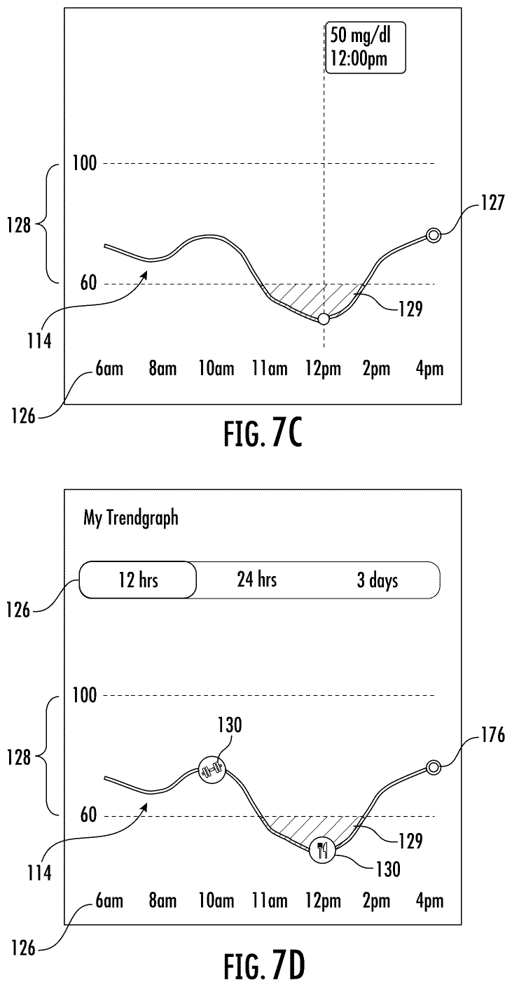

FIGS. 7A-7D show front views of a display or portion thereof with graphical user interfaces for glucose monitoring, in accordance with some aspects. The trend graph 114 is displayed via the graphical user interface on the display 108 and depicts glucose levels over a specified duration of time 126, which may be three hours, 10 hours, 12 hours or 24 hours in some examples. In some aspects, the processor 109 generates a bubble 127 on the trend graph 114 at a position corresponding to the current glucose level and a recent time of the plurality of glucose levels. The bubble 127 on the graph represents a current glucose level. A visual property of the bubble 127, including color or shading, may be generated to indicate whether the current glucose level is within, above, or below a predefined target glucose range. This provides an immediate visual cue for whether the glucose level is within, above, or below the target range. As the glucose range changes, the shading of bubble 127 may adjust accordingly, using different shading or colors to represent the current glucose range.

The predefined target glucose range 128 may define a “normal” or in-range glucose level for the specific user. The predefined target glucose range 128 may be, in some examples, 90 to 130 mg/dL, 80 to 150 mg/dL or 70 to 160 mg/dL. A region 129 above or below the predefined target glucose range 128 may be shaded with a color when glucose levels are outside the predefined range. This allows the user to easily interpret both the duration and extent of time spent above or below range, thereby supporting more effective glucose management.

FIG. 7A depicts a three-hour trend graph 114 showing regions 129 below the predefined target glucose range 128. FIG. 7B is a trend graph 114 with a time range from 6 am to 4 pm showing a region 129 above the predefined target glucose range 128. FIG. 7C depicts a trend graph 114 with a time range from 6 am to 4 μm and illustrates a region 129 shaded below the predefined target glucose range 128. The user may interact with the graph to select a specific time point in order to view the exact glucose level reading corresponding to that time. FIG. 7D is a trend graph 114 with a 12 hour range and illustrates a region 129 shaded below the predefined target glucose range 128. In some aspects, event icons 130 may be displayed on the trend graph 114 to provide insights for the impact of specific events on glucose levels. These event icons 130 help users correlate changes in their glucose levels with activities or occurrences, such as meals (fork/knife icon), exercise (barbell icon), or medication administration, thereby offering a clearer understanding of how these factors influence their glucose management. These metrics provide the user with a quick glance of the glucose level's history, without overwhelming the user with details.

For example, the trend graph 114 is displayed via the graphical user interface on the display 108 showing the plurality of glucose levels over a specified duration of time. The processor 109 may receive or determine a predefined target glucose range for a user. Based on this, one or more regions of the trend graph 114 corresponding to time intervals during which the glucose levels fall outside the predefined target glucose range is shaded. In some examples, one or more event icons 130 on the trend graph 114 is displayed via the graphical user interface, at times associated with the event. The event icons 130 represent user activity or therapy events including food intake, physical activity, or medication administration.

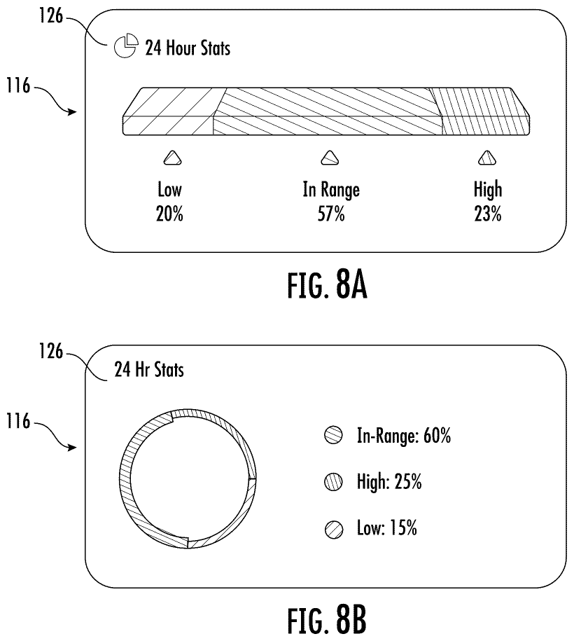

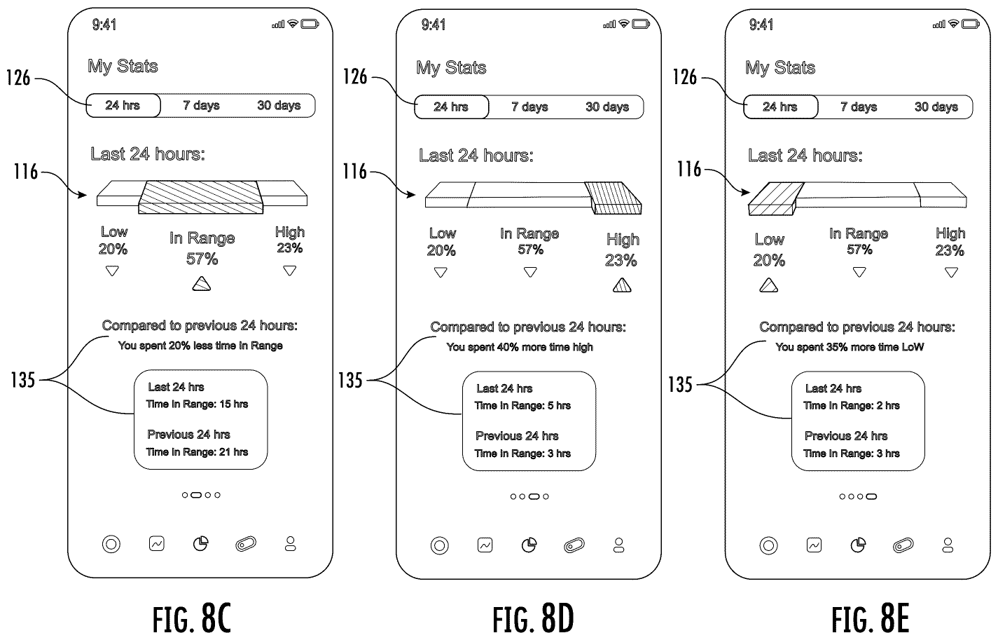

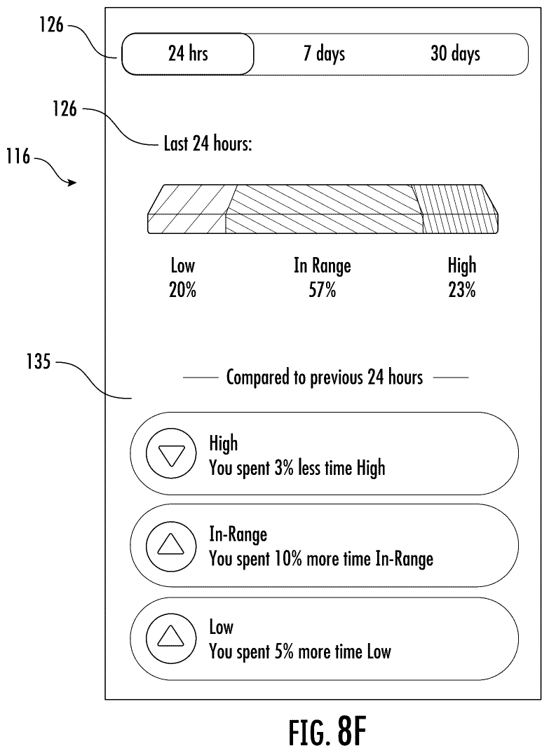

FIGS. 8A-8F show front views of a display or portion thereof with graphical user interfaces for glucose monitoring, in accordance with some aspects where further advantageous visual indicators are described. A time-in-range indicator 116 is configured to display glucose level statistics over a specified duration of time 126, such as 24 hours, 7 days, or 30 days. As illustrated in FIG. 8A, the time-in-range indicator may be presented as a rectangular graphic extending horizontally or vertically, and as shown in FIG. 8B, it may alternatively be displayed as a circular graphic. These graphics visually depict segments representing the percentage of time that glucose levels were within, below, or above a predefined target glucose range during the specified period. The segments may be color-coded to correspond to low, in-range, and high glucose categories, consistent with the visual conventions used on other screens of the application. The graphical layout provides an easy comparison of values along a linear scale, allowing users to quickly evaluate their overall glucose control and trends. The time-in-range indicator 116 can be used to track progress towards personal goals and to facilitate data comparisons over time, enhancing the user's ability to manage their diabetes effectively. For example, the processor 109 may generate a graphical time-in-range indicator 116 on the graphical user interface. The time-in-range indicator 116 visually represents, for a specified duration of time, the percentage of time during which the glucose levels were below, within, or above a predefined target glucose range. The time-in-range indicator 116 may be displayed as a graphical element having a selectable format, such as a rectangular, oval, or circular configuration, and may include visually distinguishable segments, such as color-coded regions, corresponding to low, in-range, and high glucose level categories.

FIGS. 8C-8F present additional data available to the user regarding the time spent (e.g., duration of time 126) in various glucose ranges, including low, in-range, and high. In some aspects, the user may select a specific range, causing that portion of the graphic to be highlighted and “pop out” from the other segments, providing a clear and focused view. The user can utilize this data to gain deeper insights into their glucose level patterns and make informed decisions about their diabetes management. This feature allows for more precise adjustments to medication, diet, and lifestyle, ultimately supporting improved glycemic control and health outcomes.

Referring to FIGS. 8C-8F, algorithms may be implemented to visualize data 135 as a comparison between “last data” and “previous data”. For instance, the last 24 hours can be compared to a previous 24-hour period, or the last seven days can be compared to a previous seven-day period for time spent in ranges of low/in-range/high categories. This allows users to assess the impact of health adjustments, such as changes in medication, exercise, or diet. By comparing glucose levels before and after an adjustment over equivalent time frames, users can evaluate the effectiveness of their changes. This method provides a clear and data-driven way to determine how specific adjustments influence glucose levels, facilitating better-informed decisions for diabetes management. For example, the processor 109 may process the glucose level data to compare a first time-in-range value corresponding to a recent time period to a second time-in-range value corresponding to a preceding time period of equivalent duration. On the graphical user interface of the display 108, a comparative visual indicator such as text is generated based on the comparison.

The aspects disclosed herein provide a quick and easy way to understand glucose levels at a glance. For visually impaired users, data fields can be made selectable or voice-driven, enabling the data to be read aloud to the user. This ensures that all users, regardless of visual ability, can easily access and interpret their glucose level information.

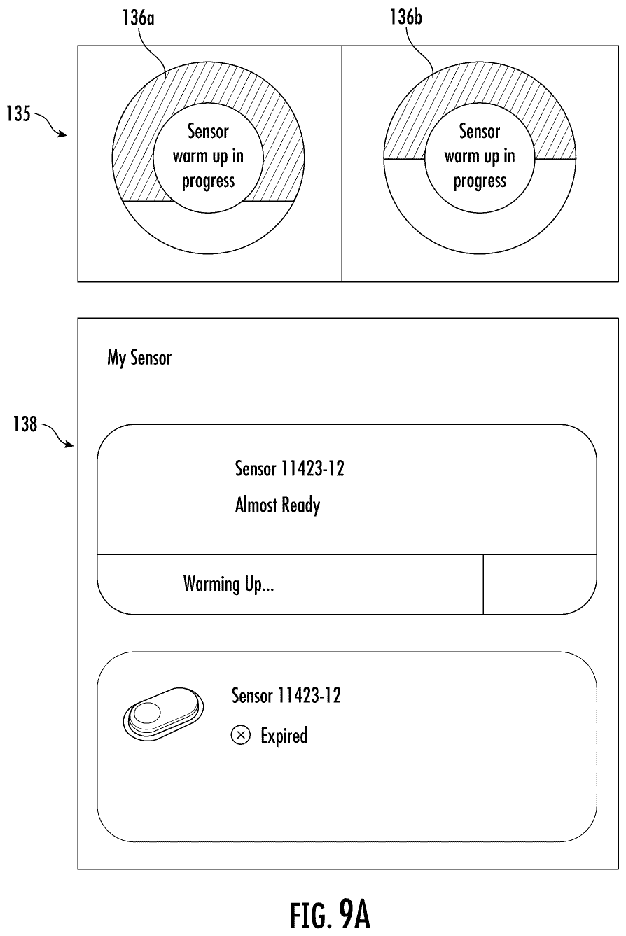

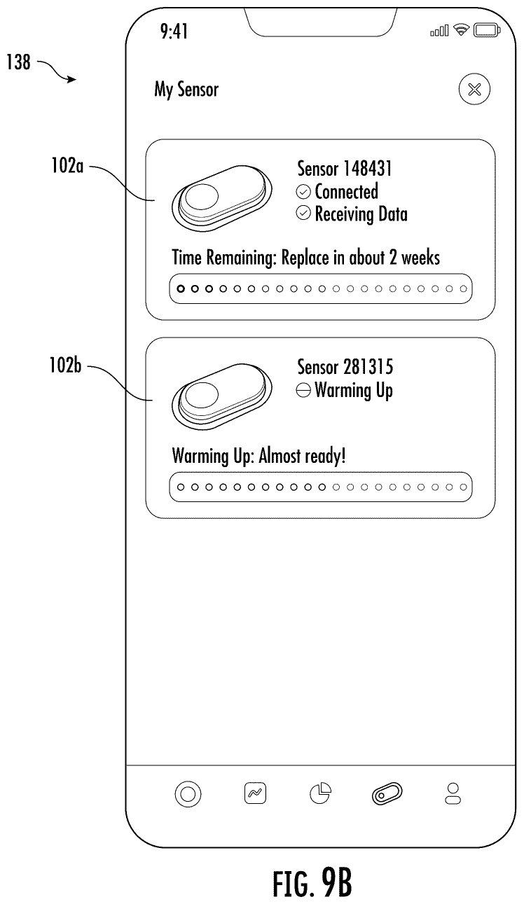

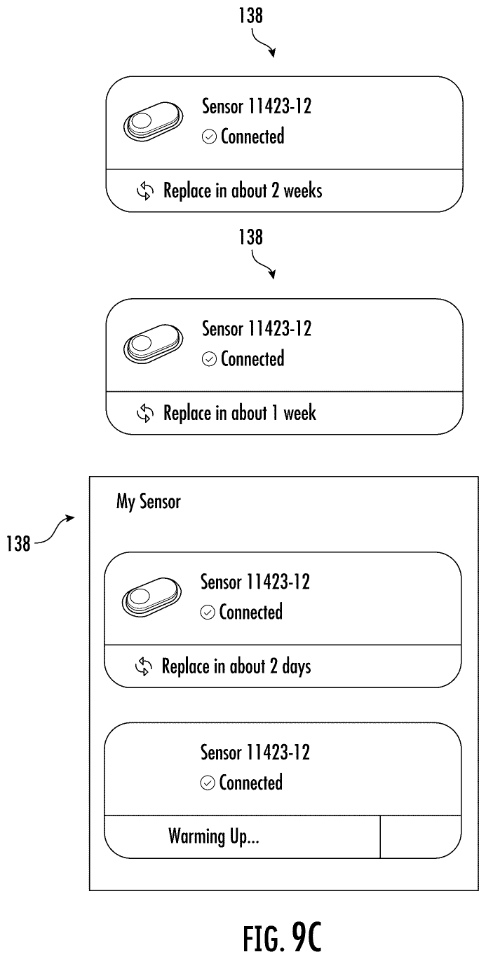

FIGS. 9A-9C are front views of a display or portion thereof with graphical user interfaces for glucose monitoring, in accordance with some aspects. FIG. 9A illustrates the display 108, presented via the graphical user interface, for data visualization in continuous glucose monitoring. The display includes information related to the connected sensors 102. Sensor status 135 communicates the operational status of the sensor 102 to the user when it is turned on. For instance, a sensor icon 136 may be gradually shaded to indicate the sensor's power-up process. In some examples, the sensor icon 136 can be represented as a circular graphic, a bar graphic, or a similar visual representation. Additionally, shading of the sensor icon 136 may be a progression providing a visual cue to the user, indicating the sensor's 102 readiness and current state. A panel 138 may also be included listing information for connected sensors 102 and expired sensors.

FIGS. 9B and 9C are further examples of the panel 138. In some aspects, the user may be wearing a first sensor 102a, and it may be time to replace it with a new sensor 102b. This may be required based on usage duration, performance, or system notification. The system 100 enables a new sensor 102b to warm up while a previous sensor 102a remains active. Once the warmup is complete, sensor 102a and sensor 102b are connected to the system 100, and the system 100 seamlessly transitions to the new sensor 102b prompting the user to remove the previous sensor 102a. This ensures continuous glucose monitoring without interruption, enhancing the user experience and maintaining consistent data collection. In some aspects, while sensor 102a and sensor 102b are connected and overlapping, the new sensor 102b can learn from the previous sensor 102a thus enhancing the performance of the new sensor 102b.

Referring to FIG. 9C, in some aspects, the system 100 provides health information about the sensor 102, including notifications on when it is time to replace the sensor 102. This ensures users are informed and can maintain optimal sensor performance for accurate glucose monitoring. For example, the processor 109 receives glucose data from a first connected glucose sensor, and simultaneously, from a second connected glucose sensor while the first connected glucose sensor remains active. One or more performance characteristics derived from the glucose data of the first connected glucose sensor is transferred to the second connected glucose sensor. A notification prompting removal of the first connected glucose sensor upon determining that the second connected glucose sensor has become active is displayed on the display 108 for the user.

In some aspects, historical data is stored and accessible to the user at any time. Users can generate detailed reports from this data, allowing for in-depth analysis and tracking of trends over time. These reports can be customized to highlight specific metrics, patterns, and insights, providing valuable information for managing and understanding the data.

Any method (also referred to as a “process” or an “approach”) described or otherwise enabled by the disclosure herein may be implemented by hardware components (e.g., machines), software modules (e.g., stored in machine-readable media), or a combination thereof. By way of example, machines may include one or more computing device(s), processor(s), controller(s), integrated circuit(s), chip(s), system(s) on a chip, server(s), programmable logic device(s), field programmable gate array(s), electronic device(s), special purpose circuitry, and/or other suitable device(s) described herein or otherwise known in the art. One or more non-transitory machine-readable media embodying program instructions that, when executed by one or more machines, cause the one or more machines to perform or implement operations comprising the steps of any of the methods described herein are contemplated herein. As used herein, machine-readable media includes all forms of machine-readable media (e.g., one or more non-volatile or volatile storage media, removable or non-removable media, integrated circuit media, magnetic storage media, optical storage media, or any other storage media, including RAM, ROM, and EEPROM) that may be patented under the laws of the jurisdiction in which this application is filed, but does not include machine-readable media that cannot be patented under the laws of the jurisdiction in which this application is filed.

Systems that include one or more machines and one or more non-transitory machine-readable media are also contemplated herein. One or more machines that perform or implement, or are configured, operable, or adapted to perform or implement operations comprising the steps of any methods described herein are also contemplated herein. Method steps described herein may be order independent and can be performed in parallel or in an order different from that described if possible to do so. Different method steps described herein can be combined to form any number of methods, as would be understood by one of ordinary skill in the art. Any method step or feature disclosed herein may be omitted from a claim for any reason. Certain well-known structures and devices are not shown in figures to avoid obscuring the concepts of the present disclosure. When two things are “coupled to” each other, those two things may be directly connected together, or separated by one or more intervening things. Where no lines or intervening things connect two particular things, coupling of those things is contemplated in at least one embodiment unless otherwise stated. Where an output of one thing and an input of another thing are coupled to each other, information sent from the output is received in its outputted form or a modified version thereof by the input even if the information passes through one or more intermediate things. Any known communication pathways and protocols may be used to transmit information (e.g., data, commands, signals, bits, symbols, chips, and the like) disclosed herein unless otherwise stated. The words comprise, comprising, include, including and the like are to be construed in an inclusive sense (i.e., not limited to) as opposed to an exclusive sense (i.e., consisting only of). Words using the singular or plural number also include the plural or singular number, respectively, unless otherwise stated. The word “or” and the word “and” as used in the Detailed Description cover any of the items and all of the items in a list unless otherwise stated. The words some, any and at least one refer to one or more. The terms may or can are used herein to indicate an example, not a requirement—e.g., a thing that may or can perform an operation, or may or can have a characteristic, need not perform that operation, or have that characteristic in each embodiment, but that thing performs that operation or has that characteristic in at least one embodiment. Unless an alternative approach is described, access to data from a source of data may be achieved using known techniques (e.g., requesting component requests the data from the source via a query or other known approach, the source searches for and locates the data, and the source collects and transmits the data to the requesting component, or other known techniques).

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific examples of the invention, it will be appreciated that those skilled in the art, upon attaining an understanding of the foregoing, may readily conceive of alterations to, variations of, and equivalents to these aspects. For instance, features illustrated or described as part of one aspect may be used with another aspect to yield a still further aspect. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A method for continuous glucose monitoring, comprising:
receiving, by a processor of a computing device operatively coupled to or integrated with a display, data comprising a plurality of glucose levels, each glucose level of the plurality of glucose levels being associated with a corresponding time;
processing, by the processor, the data to generate:
a first graphical element indicative of a current glucose level; and
one or more second graphical elements indicative of a rate and a direction of change of the glucose level;
displaying, by the processor via a graphical user interface, the first graphical element on the display; and
displaying, by the processor via the graphical user interface, the one or more second graphical elements on the display, the one or more second graphical elements being positioned relative to the first graphical element to visually indicate whether the glucose level is stable, increasing, or decreasing.

2. The method of claim 1, wherein the processing further comprises:
calculating the rate of change of the glucose level over a predefined time interval from the data;
determining that the glucose level is stable when the calculated rate of change is within a defined stability threshold range;
determining that the glucose level is increasing when the calculated rate of change is greater than an upper boundary of the stability threshold range; and
determining that the glucose level is decreasing when the calculated rate of change is less than a lower boundary of the stability threshold range.

3. The method of claim 2, wherein:
the one or more second graphical elements are equally spaced from one another and from the first graphical element when the glucose level is stable; and
the one or more second graphical elements are repositioned and animated by the processor in response to the calculated rate of change and acceleration of the glucose level.

4. The method of claim 2, wherein a quantity of the one or more second graphical elements corresponds to the calculated rate of change of the glucose level such that a greater quantity of second graphical elements indicates a faster rate of change.

5. The method of claim 4, wherein the processing further comprises:
generating a first derivative and a second derivative of glucose level over time; and
modifying the quantity of the one or more second graphical elements and animation characteristics based on the computed derivatives.

6. The method of claim 1, wherein the processing further comprises:
comparing the current glucose level to a predefined target glucose range;
determining that the glucose level is in-range when the glucose level is within the predefined target glucose range;
determining that the glucose level is low when the glucose level is less than the predefined target glucose range; and
determining that the glucose level is high when the glucose level is greater than the predefined target glucose range.

7. The method of claim 6, wherein the first graphical element is an icon rendered in a color or pattern indicative of whether the glucose level is within the predefined target glucose range, greater than the predefined target glucose range, or less than the predefined target glucose range.

8. The method of claim 1, wherein the one or more second graphical elements are arcs.

9. The method of claim 1, wherein the one or more second graphical elements are positioned above the first graphical element to indicate an increasing glucose level or below the first graphical element to indicate a decreasing glucose level.

10. The method of claim 1, further comprising animating the one or more second graphical element in a sequential manner to visually represent glucose acceleration.

11. The method of claim 1, further comprising animating the one or more second graphical elements by:
sequentially adjusting a position of each second graphical element of the one or more second graphical elements to vary spacing between adjacent second graphical elements.

12. The method of claim 1, further comprising animating the one or more second graphical elements by:
modifying a visual property of each second graphical element of the one or more second graphical elements in a time-based sequence that simulates a spring-like or ripple motion away from or toward the first graphical element, wherein the visual property is chosen from color, spacing, pulsing rate, and brightness;
wherein the animating is controlled by the processor based on one or more calculated metrics derived from the glucose data, including a rate of change in the glucose level and an acceleration of the glucose level.

13. The method of claim 12, wherein the processing further comprises:
transitioning the visual property of each second graphical element of the one or more second graphical elements in a sequence to represent a trend in glucose level over time.

14. The method of claim 12, wherein the animating further comprises:
transitioning sequentially the one or more second graphical elements to fade-to-bright and fade-to-dark across the one or more second graphical elements during outward and return movements, respectively.

15. The method of claim 12, wherein the animating is configured to move each second graphical element of the one or more second graphical elements in a time-ordered ripple sequence that visually conveys a change in acceleration of the glucose level.

16. The method of claim 12, wherein the pulsing rate of each second graphical element of the one or more second graphical elements increases with an increasing magnitude of glucose acceleration.

17. A system for continuous glucose monitoring comprising:
a computing device operatively coupled to or integrated with a display, and having a processor and memory, the processor configured to execute instructions to:
receive data comprising a plurality of glucose levels, each glucose level of the plurality of glucose levels being associated with a corresponding time;
process the data to generate:
a first graphical element indicative of a current glucose level; and
one or more second graphical elements indicative of a rate and a direction of change of the glucose level;
display via a graphical user interface, the first graphical element on the display; and
display via the graphical user interface, the one or more second graphical elements on the display, the one or more second graphical elements being positioned relative to the first graphical element to visually indicate whether the glucose level is stable, increasing, or decreasing.

18. The system of claim 17, wherein the processor is further configured to execute instructions in the memory to:
calculate the rate of change of the glucose level over a predefined time interval from the data;
determine that the glucose level is stable when the calculated rate of change is within a defined stability threshold range;
determine that the glucose level is increasing when the calculated rate of change is greater than an upper boundary of the stability threshold range; and
determine that the glucose level is decreasing when the calculated rate of change is less than a lower boundary of the stability threshold range.

19. The system of claim 17, wherein:
the one or more second graphical elements are equally spaced from one another and from the first graphical element when the glucose level is stable; and
the one or more second graphical elements are repositioned and animated by the processor in response to the calculated rate of change and acceleration of the glucose level.

20. The system of claim 17, wherein the processor is further configured to execute instructions in the memory to:
animate each second graphical element of the one or more second graphical elements by:
sequentially adjusting a position of each second graphical element of the one or more second graphical elements to vary spacing between adjacent second graphical elements; and
modifying a visual property of each second graphical element of the one or more second graphical elements in a time-based sequence that simulates a spring-like or ripple motion away from or toward the first graphical element, wherein the visual property includes color, spacing, pulsing rate, and brightness;
wherein the animating is controlled by the processor based on one or more calculated metrics derived from the glucose data, including the rate of change in the glucose level and the acceleration of the glucose level.
